(19) United States
(12) Patent Application Publication
(10) Pub. No.: US 2012/0184545 A1
(43) Pub. Date: Jul. 19, 2012

(54) HEART-SLOWING DRUG CONTAINING SHORT-ACTING BETA-BLOCKER AS TEH ACTIVE INGREDIENT

(75) Inventors: Masaharu HIRANO, Tokyo (JP); Kenzo Nakao, Osaka-shi (JP); Ken Mizushima, Osaka (JP); Morito Akisada, Tokyo (JP); Takahiro Azuma, Tokyo (JP)

(73) Assignee: ONO PHARMACEUTICAL CO., LTD., Osaka (JP)

(21) Appl. No.: 13/432,160

(22) Filed: Mar. 28, 2012

Related U.S. Application Data
(63) Continuation of application No. 10/567,569, filed on Feb. 8, 2006.

(30) Foreign Application Priority Data
Aug. 8, 2003 (JP) 2003-290301

(51) Int. Cl. A61K 31/5377; A61P 9/00

(52) U.S. Cl. 514/231.5

(57) ABSTRACT

The present invention relates to an agent which slows down the heart rate which has an excellent controlling ability in diagnostic imaging comprising a short-acting β-blocker (e.g. landiolol hydrochloride or esmolol hydrochloride). The short-acting β-blocker has a property of slowing down the heart rate and it can temporarily suppress the tachycardia at diagnosis. According to the dose and the method of administration, it can control the period for the heart rate adjustment. Also, the present invention relates to a diagnostic imaging auxiliary comprising a short-acting β-blocker as active ingredient.

### CROSS REFERENCE TO RELATED APPLICATIONS

This is a continuation application of U.S. application Ser. No. 10/567,569 filed Feb. 8, 2006, which is a National Stage of PCT/JP2004/011672 filed Aug. 6, 2004, which claims benefit of Japanese Application No. 2003-290301 filed Aug. 8, 2003. The entire discloses of the prior applications are incorporated herein by reference.

### TECHNICAL FIELD

The present invention relates to an agent which slows down the heart rate, comprising a short-acting β-blocker as active ingredient.

More specifically, the present invention relates to an agent which slows down the heart rate in diagnostic imaging, comprising a short-acting β-blocker as active ingredient.

The present invention also relates to an assisting agent for diagnostic imaging, comprising a short-acting β-blocker as active ingredient.

### BACKGROUND ART

It is said that, two inspections are required to diagonize ischemic heart diseases conclusively, i.e. “a stress myocardial scintigram inspection” for the purpose of detecting an ischemic site of a coronary artery and “a heart catheter inspection (coronary angiography inspection)” for detecting a stenosed site of a coronary artery. Since among them, the heart catheter inspection costs too much and it can be performed only by professional doctors and it includes a risk of death due to complications. Therefore, less expensive, easily performable in the out-patient clinic and non-invasive inspections have been hoped for.

In such situations, coronary angiography which uses multi-slice helical computed tomography (MSCT), which is also called multi-detector helical computed tomography (MDCT), it is abbreviated as MSCT hereafter, fulfills these conditions and it is remarked as an inspection method which replaces the heart catheter inspection.

With the images given from a conventional CT, only a diagnosis by planar images vertical to the body axis can be performed, while MSCT can photograph more planar images in a short time because of photographing plural planar images at the same time, and thereby it is possible to reconstitute three-dimensional images. Therefore, it is possible to detect the stenosis site of a coronary artery based on the images by constituting images of a coronary artery which runs in three dimensions and complicatedly. However, since MSCT has low temporal resolution, it is subject to the heart rate and respirations.

Therefore, at present, in the medical field, sharp images are obtained by slowing down the heart rate as a result of administration of oral β-blockers (see Journal of Japan Radiological Society, 1993, Vol. 53, No. 9, 1033-1039 and The Japanese Journal of Acute Medicine, 2003, Vol. 27, 719-725).

However, existing oral β-blockers have some troubles; i.e. (1) it takes some time to show the drug effect and to disappear the drug effect, and therefore it takes long time from administration to inspection, and observation after inspection, (2) if there is individual distinction in the time until the effect of the drug arises and it takes some time to onset of the drug effect, the inspection will be troublesome, (3) it is hard to adjust the dose because of bad controlability in the heart rate, (4) there is fear of post-operative safety (e.g. lowering of blood pressure etc. because of continuation of β-blocking effect), etc.

Since it takes 10 to 15 minutes to adjust the heart rate in MSCT diagnosis, the patients who will have an inspection cannot move safely after inspection if the slowing down of the heart rate continues too long. The situation like this is not necessarily preferable for the patients.

Therefore, a drug which excellently adjusts various kinds of diagnostic imaging has been hoped for the purpose of improvement of angiography such as MSCT, improvement of safety for patients, shortening of the inspection time, etc.

On the other hand, (−)-[(S)-2,2-dimethyl-1,3-dioxolan-4-yl]methyl-3-[4-[(S)-2-hydroxy-3-(2-morpholinocarbonylamino)ethylamino]propoxy]phenylpropionic acid hydrochloride (CAS registry number: 144481-98-1; called landiolol hydrochloride hereafter) is used in emergency treatment of intraoperative and tachyarrhythmia (fibrillation, atrial flutter, sinus tachycardia) as a short-acting β1-blocker (e.g. see the gazette of JP 3-72475).

Methyl 3-[4-[2-hydroxy-3-(isopropylamino)propoxy]phenyl]propionic acid hydrochloride (CAS Registry No. 103598-03-4; called esmolol hydrochloride hereafter) is used.

Landiolol hydrochloride is quickly decomposed by esterases in blood and a liver to inactive compounds and it has an extremely short half life in blood of approximately 4 minutes, compared with existing β-blockers. The selectivity on β₁ (β₁/β₂) is approximately 250 and so it has a high selectivity on a heart, and it is to be estimated it has little influence on airway system.

And since esmolol hydrochloride also has a short half life in blood of approximately 9 minutes, the same effect will be expected.

### DISCLOSURE OF THE INVENTION

As a result of energetic investigation in view of the above situations, the present inventors have found out that a short-acting β-blocker, surprisingly, shows an excellent effect as an agent which slows down the heart rate in diagnostic imaging including MSCT as active ingredient.

A short-acting β-blocker like this is especially useful in the short-term diagnosis because it is easy to control the heart rate in the use of infused intravenous administration. Compared with the existing pharmaceutical agents, for example, landiolol hydrochloride has a very short period of a half life (3 to 5 minutes) in the blood, therefore, it is possible to adjust the heart rate by changing the dose and the method of administration.

Hereby use of a short-acting β-blocker having convenience in controlability avoids the problem of existing β-blockers, improves the ability of angiography by MSCT, and consequently avoids adverse effects (staggering, vertigo, etc.) after angiography of the patients.

The present invention relates to

1. an agent which slows down the heart rate comprising a short-acting β-blocker,
2. the agent which slows down the heart rate according to above 1, wherein the half life of the short-acting β-blocker in a human is 2 to 20 minutes,
3. the agent which slows down the heart rate according to above 1, wherein the heart rate lowering agent is landiolol hydrochloride or esmolol hydrochloride,
4. the agent which slows down the heart rate according to above 1, which is useful for diagnostic imaging,
5. the agent which slows down the heart rate according to above 4, wherein the diagnostic imaging is by nuclear angiography, magnetic resonance imaging diagnosis or echography,
6. the agent which slows down the heart rate according to above 1, which is combined with at least one selected from anesthetic adjunct, muscular relaxant and anxiolytic,
7. the agent which slows down the heart rate according to above 3, which is combined with a contrast medium,
8. the agent which slows down the heart rate according to above 7, wherein the contrast medium is an iodinated contrast medium, a borated contrast medium, a xenon contrast medium, a barium contrast medium,
9. the agent which slows down the heart rate according to above 3, which is useful for diagnostic imaging by X-ray angiography using multi-slice helical CT,
10. a method for slowing down the heart rate, which comprises administering to a mammal an effective amount of a short-acting β-blocker,
11. use of a short-acting β-blocker for the manufacture of an agent which slows down the heart rate,
12. a diagnostic imaging auxiliary comprising a short-acting β-blocker,
13. the diagnostic imaging auxiliary according to above 12, wherein the half life in a human of the short-acting β-blocker is 2 to 20 minutes,
14. the diagnostic imaging auxiliary according to above 12, wherein the short-acting β-blocker is landiolol hydrochloride or esmolol hydrochloride,
15. the diagnostic imaging auxiliary according to above 12, which is a diagnostic imaging auxiliary for a heart, coronary arteries, kidneys, a liver, a uterus, a stomach, intestines, lungs or thoracic aortas,
16. the diagnostic imaging auxiliary according to above 12, which is useful for diagnosing heart diseases,
17. a method for assisting diagnostic imaging, which comprises administering to a mammal an effective amount of a short-acting β-blocker,
18. use of a short-acting β-blocker for the manufacture of a diagnostic imaging auxiliary,
19. the agent which slows down the heart rate according to above 1, which is a liquid formulation,
20. the agent which slows down the heart rate according to above 19, which is an aqueous liquid formulation and
21. the agent which slows down the heart rate according to above 19 or 20 wherein the short-acting β-blocker is landiolol hydrochloride.

That is, the present invention relates to an agent which slows down the heart rate, characterized by administering to a patient who goes through diagnostic imaging.

Specifically, the present invention relates to an agent which slows down the heart rate, characterized by using a short-acting β-blocker optionally together with an contrast medium.

The diseases in which a short-acting β-blocker is used are for example, visceral diseases such as heart diseases, liver diseases, digestive organ diseases.

Heart diseases include, ischemic heart diseases (e.g. angina pectosis, cardiac infarction, etc.), arrhythmia, etc. and ischemic heart diseases are preferable.

Short-acting β-blockers include, for example, landiolol hydrochloride and esmolol hydrochloride and do not limit to them but include those which will be found out in the future. For example, short-acting β-blockers include the compounds which has a half life of approximately 2 to 20 minutes in human blood and more preferably approximately 3 to 10 minutes.

In the present invention, short-acting β-blockers may be used alone or they may be used with e.g. muscle relaxant agents, antianxiety agents, anesthetic adjuncts, etc.

Muscle relaxant agents used in the present invention to be used in combination with a short-acting β-blocker include, e.g. botulinus toxin type A, papaverine hydrochloride, dantrium, dantrolene sodium, vecuronium bromide, pancuronium bromide, suxamethonium chloride, etc.

Antianxiety agents used in the present invention to be used in combination with a short-acting β-blocker include, e.g. diazepam, oxazolam, flutazolam, alprazolam, ethyl loflazepate, tofisopam, etizolam, bromazepam, clotiazepam, lorazepam, etc.

Anesthetic adjuncts used in the present invention to be used in combination with a short-acting β-blocker include, e.g. pethidine, fentanyl, dromoran, etc.

In the present invention, the heart rate may be controlled by adjusting the dose and the method for administration of the short-acting β-blocker minutely. Thereby, necessary and sufficient effect will be drawn in the diagnostic imaging.

Specifically, for example, the dose of landiolol hydrochloride is preferably, (i) after performing a sustained intravenous administration of a high dose, and then (ii) performing a sustained intravenous administration of a low dose.

For example, the high dose for the bolus (swift intravenous) administration for a short period in the process (i) is, a necessary dose in order to acquire steady blood in the introduction of landiolol hydrochloride. Specifically, approximately 0.0315 to 0.250 mg/minute per 1 kg of the patient, particularly preferably, approximately 0.063 to 0.125 mg/minute per 1 kg of the patient. The short period in the process (i) is the time in order to acquire the steady blood concentration in the introduction of landiolol hydrochloride. Particularly, approximately 30 seconds to 3 minutes, more preferably approximately 30 seconds to 2 minutes. Most preferable is approximately 1 minute.

The swift intravenous administration in the process (i) is to administer the above dose for a short period, e.g. an administration using an infusion pump, a volumetric pump, etc. or manual administration.

The low dose in the process (ii) for the infusion (sustained intravenous) administration is, a sufficient dose for slowing down the steady heart rate of the patient who goes through a CT inspection. Specifically, approximately 0.01 to 0.08 mg/minute per 1 kg of the patient. More preferably, approximately 0.02 to 0.04 mg/minute per 1 kg of the patient. The long period in the process (ii) is, a sufficient time for acquiring the lowering of the steady heart rate of the patient who goes through a CT inspection. Preferably, approximately 5 to 20 minutes, and more preferably, approximately 10 minutes.

The sustained intravenous administration in the process (ii) is to administer the above dose for a long period, e.g. an administration using an infusion pump, a volumetric pump, etc. or manual administration.

In the present invention, the purpose of swift intravenous administration of a high dose in the process (i) is to upper the blood concentration of landiolol hydrochloride immediately, while the purpose of intravenous administration of a low dose in the process (ii) is to maintain the blood concentration of landiolol hydrochloride, whose half life is short.

“Swift intravenous administration” means administration of a high dose above for a short period and it has the same meaning as bolus administration. “Sustained intravenous administration” means administration of a low dose above for a long period and it has the same meaning as infusion administration.

The ratio of the “high dose in the process (i)” and the “low dose in the process (ii)” is preferably approximately 2:1 to 5:1, provided that the process (ii) is done following the process (i), and more preferably approximately 2:1 to 4:1, and particularly preferably approximately 3:1.

The doses of the second process (i) and (ii) to be administered followed by the first process (i) and (ii) have higher amount than the first process (i) and (ii) respectively.

When the combination administration of the process (i) and (ii) is done once, (1) (i) approximately 0.063 mg/kg/minute→(ii) approximately 0.02 mg/kg/minute or (2) (i) approximately 0.125 mg/kg/minute→(ii) approximately 0.04 mg/kg/minutes preferable and particularly (1) is more preferable.

When the combination administration of the process (i) and (ii) is done twice, (i) approximately 0.063 mg/kg/minute→(ii) approximately 0.02 mg/kg/minute→(i) approximately 0.125 mg/kg/minute→(ii) approximately 0.04 mg/kg/minute is preferable.

Other than those above, landiolol hydrochloride may be administered by easier method. For example, it is also effective; one ampoule (50 mg) of a freeze-dried formulation of landiolol hydrochloride (brand name: ONOACT 50 (manufactured by Ono Pharmaceutical Co., Ltd.) is dissolved in saline (20 ml) and 1 ml of the resulting solution is administered 2-3 times approximately every 20 minutes (i.e. 2.5 mg), and while confirming its effect, the solution is equipped in a syringe pump to adjust the dose to be administered by intravenous injection.

Particularly, in the severe patients, it is safer to adjust from the low dose under examining the change of the heart rate.

The heart rate to be adjusted by the compounds used in the present invention is preferably, the heart rate wherein a sharp image is given in the diagnostic imaging and no excess bradycardia is given; i.e. particularly 45 to 65 beats per minute, more preferably 50 to 60 beats per minute.

In the present invention, for diagnostic imaging, X-ray angiography such as MSCT etc., radiation angiography such as PET (positron-emission tomography), scintigraphy (cardiac muscle, heart function, liver, etc.), etc., MRI (magnetic resonance imaging) diagnosis such as MRA (magnetic resonance angiography) etc., ultrasound diagnostic imaging, etc. are preferably used, but the present invention is not limit to them.

In the radiation angiography, radiant ray is preferably X-ray, α-ray, β-ray, γ-ray, etc. and particularly preferable is X-ray.

In the present invention, the target organs for angiography include, for example, a heart, coronary arteries, kidneys, a liver, a uterus, a stomach, intestines, lungs and thoracic aortas, etc. and are not limited to them. More preferably are a heart or coronary arteries, and coronary arteries are most preferable.

The short-acting β-blocker in the present invention is optionally used with contrast media.

Contrast media are not limited in particular, but for example, iodinated contrast media (e.g. amidotrizoic acid, ioxaglic acid, ioxilan, iotalamic acid, meglumine iotroxate, iotrolan, iopanoic acid, iopamidol, iopromide, iohexyl, iomeprol, sodium iopodate, metrizoic acid, iodamide, iodoxamic acid, iodine addition products of the ethyl esters of the fatty acid obtained from poppyseed oil, etc.), xenon contrast media (e.g. xenon gas, xenon injection solution, etc.), barium contrast media (e.g. barium sulfate etc.), ferreous contrast media (e.g. ferumoxides, iron ammonium citrate, etc.), gadolinium contrast media (e.g. meglumine gadopentetate, gadoteridol, etc.).

Radioactive isotopes used in radiocontrast angiography include, e.g. hydrogen, carbon, nitrogen, oxygen, fluorine, technetium, thallium, iodine, etc. Particularly, thallium chloride (201TlCl), meta-iodo-benzylguanidine (123I-MIBG), carbon dioxide, carbon monooxide, oxygen, fluoroglucose, hydrogen cyanide, etc.

In PET (positron-emission tomography), ultra short-lived radionuclides (11C, 13N, 15O, 18F, etc.) may also be preferably used.

Tomographic MSCT equipments in the present invention, for example, include the followings; Aquilion 16, Aquilion 8. Aquilion 4, Aquilion/multi, Asteion/dual (above are manufactured by Toshiba), IDT16 (manufactured by Philips Medical Systems), Sensation Cardiac, Sensation 16, Sensation 10, Emotion 6, Volume class/sensation 4, Emotion Dio (manufactured by Siemens AG), ROBUSTO series (manufactured by Hitachi Medical Corporation), Mx8000 (manufactured by Philips), LightSpeed Ultra 16, LightSpeed Ultra, LightSpeed Plus/Qx/i series, HiSpeed QX/i, HiSpeed NX/i series, ProSpeed FII (manufactured by GE Yokogawa Medical System), etc.

As the numbers of the detector rows in the tomographic equipment, 4-slices, 8-slices, 16-slices, 32-slices and 64-slices are all preferable, and as the slices increase in number, the image becomes sharper, and as the time for inspection is shortened, 16-slices, 32-slices and 64-slices are more preferable, therefore.

In the present invention, β-blockers include those compounds which antagonize β₁, β₂ and β₃ receptors, and the compound which acts on heart specifically, i.e. the compound which acts on β₁ receptor specifically is preferable.

Landiolol hydrochloride used in the present invention is described in JP 2,004,651(B) and JP 3,302,647(B) and its chemical name is (−)-[(S)-2,2-dimethyl-1,3-dioxolan-4-yl]methyl-3-[4-[(S)-2-hydroxy-3-(2-morpholinocarbonylamino)ethylamino]propoxy]phenylpropionic acid hydrochloride.

Landiolol hydrochloride is used in various forms of pharmaceutical composition according to known methods, e.g. JP 2,004,651(B) or JP 3,302,647(B). For example, any formulation with which intravenous administration is applied is preferable, and particularly liquid formulation and freeze-dried formulation which is dissolved in a solubilizing agent before use. In such formulations, approximately 1 to 1,000 mg of landiolol hydrochloride is preferably contained, more preferably approximately 10 to 100 mg and much more preferably approximately 50 mg.

In the pharmaceutical composition, for example, additives are selected from excipients, binding agents, moistening agents, stabilizers, etc.

Administration of the short-acting β-blockers used in the present invention is preferably injection, which is applicable to the purpose of adjusting the heart rate appropriately.

Injective formulation is preferably, liquid formulation or freeze-dried formulation which will be dissolved in an solubilizing agent before use.

Liquid agents are used by dissolving, suspending or emulsifying one active substance or more in solubilizing agents.

Solubilizing agents include, for example, distilled water for injection, vegetable oil, propylene glycol, polyethylene glycol, alcohols such as ethanol, etc. and a combination thereof.

The present formulation may further include, e.g. a stabilizing agent (e.g. sodium citrate, sodium edetate), a solubilizing agent (e.g. glutamic acid, aspartic acid, polysorbate 80 (registered trademark), etc.), a suspending agent (e.g. surfactants such as stearyl triethanolamine, sodium lauryl sulfate, lauryl aminopropionic acid, lecithin, benzalkonium chloride, benzethonium chloride, glyceryl monostearate, polyethoxylated hydrogenated castor oil, polysorbate, etc., multiple alcohols such as glycerine, macrogol, etc., sugars such as sorbitol, mannitol, sucrose, etc., celluloses such as methyl cellulose, carboxymethyl cellulose, hydroxypropylmethyl cellulose, etc., hydrophilic macromolecules such as polyvinyl alcohol, polyvinylpyrrolidone, carboxyvinyl polymer, sodium carboxymethyl cellulose, methyl cellulose, hydroxymethyl cellulose, hydroxyethylcellulose, hydroxypropyl cellulose, etc., chondroitin sulfate, etc.), an emulsifying agent (e.g. glycerine ester, saponin (sophora saponin, quillai extract, soybean saponin, etc.), sucrose fatty acid ester (e.g. sucrose ester, etc.). lecithin (e.g. vegetable lecithin, yolk lecithin, etc.), a soothing agent (e.g. benzyl alcohol, chlorobutanol, propyleneglycol, ethyl aminobenzoate, lidocaine), a buffering agent (e.g. phosphates (sodium hydrogenphosphate, sodium dihydrogen phosphate, etc.), boric acid, borax, acetate (e.g. sodium acetate etc.), a carbonate (e.g. sodium carbonate, calcium carbonate, potassium carbonate, etc.), citric acid, sodium L-glutamate, etc.), a pH adjusting agent (e.g. sodium hydroxide, potassium hydroxide, trisodium phosphate, disodium hydrogen phosphate, hydrochloric acid, nitric acid, citric acid, boric acid, acetic acid, etc.), a preserving agent (e.g. paraoxybenzoates such as propyl paraoxybenzoate, butyl paraoxybenzoate, parabens such as methylparaben, ethylparaben, propylparaben, butylparaben, etc., inverted soap such as benzalkonium chloride, benzethonium chloride, chlorhexidine gluconate, cetylpyridium chloride, etc., alcohol derivatives such as chlorobutanol, benzylalcohol, phenethylalcohol, etc., organic acids and salts thereof such as sodium dehydroacetate, sorbic acid, sodium sorbate, phenols such as parachloromethoxyphenol, parachloromethacresol, etc.), a tonicity agent (e.g. glucose, D-sorbitol, sodium chloride, glycerine, D-mannitol, potassium chloride, concentrated glycerine, propylene glycol, sucrose, etc.), etc. They are sterilized in the final process or manufactured by aseptic manipulation. Otherwise, sterile solid compositions, for example, freeze-dried products are manufactured and they may be sterilized or dissolved in sterile purified water or other solvents before use.

### BRIEF DESCRIPTION OF THE DRAWING

FIG. 1 is one example of graphic image of angiography when landiolol hydrochloride is administered.

FIG. 2 is one example of graphic image of angiography when landiolol hydrochloride is not administered.

### BEST MODE FOR CARRYING OUT THE INVENTION

The present invention is illustrated by the following examples but the present invention is not limited to them.

### Example 1

Evaluation of efficacy and safety of landiolol hydrochloride to the coronary angiography ability using MSCT:

In the imaging of MSCT, LightSpeed Ultra 16 (16-slices MSCT, manufactured by GE medical systems) was used.

As a Workstation, Advantage Workstation AW 4.2 (manufactured by GE Medical Systems) was used.

As an imaging analysis soft, CardIQ Analysis II manufactured by GE Medical Systems was used.

As contrast media, iomeprol 350 mgI/ml (brand name: Iomeron 350, manufactured by Eisai Co., Ltd., was used.

### (1) Administration of Landiolol Hydrochloride

To four angina pectoris patients, was intravenously administered landiolol hydrochloride swiftly over a period of 1 minute (0.063 mg/kg/minute), followed by a sustained intravenous administration over a period of 10 minutes (0.02 mg/kg/minute).

### (2) Coronary Angiography by MSCT (Administration of Contrast Media and Method for Imaging)

The contrast media were administered 10 minutes after confirming that the heart rate was slowed down. The imaging timing was determined by a test injection method using contrast media. As a result of swift administration of iomeprol (10 ml) and saline (20 ml) sequentially, it took approximately average of 16 seconds for iomeprol to reach heart. Afterwards, iomeprol (70 ml) and saline (20 ml) were swiftly administered sequentially and in time with the timing when iomeprol reached heart, MSCT imaging was started.

Radiographic demonstration of a coronary artery was done with (A) volume rendering (VR/three-dimensional image) method, (B) curved MPR (multi planar reformation) method and (C) vessel analysis (vessel automatic detection). Three-dimensional overall figures of coronary artery were grasped with (A), and then followed by evaluation of the lesion site with (B) and (C), optionally followed by addition of an orthogonal cross-section imaging of a coronary artery for further confirmation.

### Results:

To the above four angina pectosis patients, landiolol hydrochloride was administered. Table 1 shows the changes of the heart rate and the blood pressure of systolic blood pressure. The inhibition rate (%) was calculated by {(post-value/(pre-value)×100−100}.

TABLE 1

| | Before | After | |
|---|---|---|---|
| | administration | administration | Inhibition |
| | (n = 4) | (n = 4) | rate (%) |
| Heart rate | 61.0 | 54.8 | −10.2 |
| (/minute) | | | |
| Systolic blood | 173.3 | 161.8 | −6.6 |
| pressure (mmHg) | | | |

As is apparent from table 1, by the administration of landiolol hydrochloride, the decrease of the heart rate was confirmed. As a result, the coronary angiography by MSCT was extremely sharp and it is concluded that the ability of coronary angiography was improved accompanying the effect of slowing down the heart rate.

As is apparent from table 1, it was confirmed that the heart rate was slowed down by administration of landiolol hydrochloride. As a result, the imaging of coronary angiography by MSCT was extremely sharp and the decrease of the heart rate rendered the higher resolution.

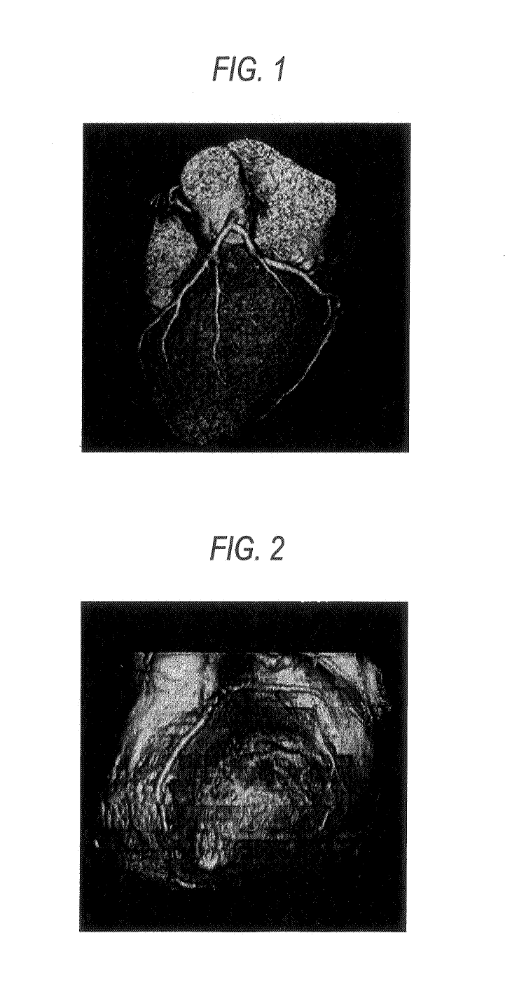

FIG. 1 shows one case of the angiography when landiolol hydrochloride was pre-administered. On the other hand, FIG. 2 shows one case of the angiography when landiolol hydrochloride was not pre-administered.

To compare these figures, FIG. 1 shows up a sharp image of coronary arteries, whereas in FIG. 2 horizontal noises are standing out in the angiography, so it is obvious that the angiography of the coronary artery is not sharp. And since no excessive lowering of the blood pressure was found, the efficacy and safety of the present drug were recognized.

From these, landiolol hydrochloride is a useful agent for the purpose of gaining a sharp coronary angiography. Moreover, since the efficacy of the drug disappears in a short period, adverse effects such as staggering or qualm do not occur resulting from continuation of the decreased heart rate after performing the imaging, resulting in minimum burden to the patients on whom the imaging was performed.

### Formulation Example 1

### Preparation of a Freeze-Dried Formulation Comprising 50 mg of Landiolol Hydrochloride

The following components were admixed by a conventional method, and the resulting solution was sterilized by a conventional method, placed 5 ml portions into vials and freeze-dried to give 10,000 vials each containing 50 mg of active ingredient.

| | |
|---|---|
| Landiolol hydrochloride | 500 g |
| D-Mannitol | 500 g |
| Sodium hydroxide | 420 mg |
| Distilled water | 6000 ml (total) |

### Formulation Example 2

### Preparation of a Liquid Formulation Comprising 50 mg of Landiolol Hydrochloride

The following components were admixed by a conventional method and the resulting solution was sterilized by a conventional method. The solution was filled in vials each 5 ml to give 10,000 vials of liquid solution each containing 50 mg of active ingredient.

| | |
|---|---|
| Landiolol hydrochloride | 500 g |
| D-Mannitol | 500 g |
| Sodium hydroxide | 420 mg |
| Distilled water | 6000 ml (total) |

### Formulation Example 3

### Preparation of a Liquid Formulation Containing 100 mg of Esmolol Hydrochloride

The following components were admixed by a conventional method, and the resulting solution was sterilized by a conventional method, placed 10 ml portions into vials to give 10,000 vials each containing 100 mg of active ingredient.

| | |
|---|---|
| Esmolol hydrochloride | 1000 g |
| Sodium acetate | 280 g |
| glacial acetic acid | 5.46 g |
| Distilled water | 100 L in total |

### INDUSTRIAL APPLICABILITY

A short-acting β-blocker is quick in expressing and disappearing the effect of the drug and it makes it possible to control the heart rate for a short period, which is necessary for coronary angiography represented by MSCT. Therefore, it can shorten the observation time considerably; waiting time from administration to expressing of the drug efficacy, from inspection to disappearing of the drug efficacy after diagnosis, compared with existing oral β-blocker. It can reduce the time burden of a patient in MSCT inspection. Also, for medical institution, they do not have to force the patient to take a medicine several hours before the diagnosis. The diagnosis is done in a short period when the patient comes to the clinic.

Unlike oral β-blockers, it is possible to control the heart rate by adjusting the dose. In particular, those patients in out-patient clinic, they can go back to their daily life soon after checkup without lingering thin pulse.

A short-acting β-blocker is excellent in controlability and therefore it is useful as a diagnostic auxiliary in diagnostic imaging.

1. A method for assisting diagnostic imaging for a heart or coronary arteries to adjust the heart rate for 10 to 15 minutes and avoid excessive lowering of the blood pressure, which comprises sustained intravenous administration of landiolol hydrochloride to a patient diagnosed for heart diseases in an amount of 0.06575 mg to 2.1 mg per 1 kg of said patient's weight in one diagnostic imaging.

2. The method according to claim 1, which comprises
a step (i) rapid intravenous administration in an amount of 0.063 mg to 0.125 mg per 1 kg of the patient's weight per minute for 1 minute and
a following step (ii) sustained intravenous administration in an amount of 0.02 mg to 0.04 mg per 1 kg of the patient's weight per minute for 10 minutes.
